(19) United States
(12) Patent Application Publication
(10) Pub. No.: US 2023/0371858 A1
(43) Pub. Date: Nov. 23, 2023

(54) Device and Method for Detection of Post-Surgical Infection and Other Disease

(71) Applicant: University of Cincinnati, Cincinnati, OH (US)

(72) Inventors: Tao Li, Mason, OH (US); Jiaxin Jiang, Cincinnati, OH (US); Krithika Sureshkumar, Cincinnati, OH (US); Chandrashekhar Choudhary, Cincinnati, OH (US); Henry Claude Sagi, Cincinnati, OH (US); Chia-Ying Lin, Mason, OH (US); Michael Archdeacon, Cincinnati, OH (US)

(21) Appl. No.: 18/201,179

(22) Filed: May 23, 2023

Related U.S. Application Data
(60) Provisional application No. 63/344,850, filed on May 23, 2022.

(51) Int. Cl. A61B 5/1473; A61B 5/00

(52) U.S. Cl. CPC A61B 5/14735; A61B 5/686

(57) ABSTRACT

A device for providing passive, wireless, in vivo detection of post-surgical infection in a surgical implant or prosthesis is provided. The device includes at least one magnetoelastic-based sensor associated with the implant or prosthesis. At least one magnetoelastic-based sensor is a differential sensor. Also, the differential sensor comprises a reference element and a sensing element.

### CROSS-REFERENCE TO RELATED APPLICATIONS

This application claims priority to, and the benefit of the filing date of, U.S. Patent Application Ser. No. 63/344,850, filed on May 23, 2022, the disclosure of which is incorporated by reference herein in its entirety.

### TECHNICAL FIELD

The present invention relates to implantable devices for detection of post-surgical infection.

### BACKGROUND OF THE INVENTION

Total knee arthroplasty (TKA), a surgical procedure to treat chronic degenerative conditions of the knee by replacement, is becoming increasingly prevalent due to an aging population. The annual number of patients receiving TKA in the US was ≈1 million in 2020 and is expected to double by 2030. Periprosthetic joint infection (“PJI”) is a devastating complication of TKA and is typically caused by bacterial contamination. PJI is also a potential complication in post-traumatic infection after the treatment of open fractures.

Despite a low but relatively stable incidence rate of ≈2%, PJI can require revision surgeries that are accompanied by substantial functional and socioeconomic burden for both the patient and society. This is further aggravated with the rapidly increasing number of TKA cases. With an average hospital cost of over $28 k per PJI case and estimated 40 k cases per year, the annual financial impact is estimated to exceed $1.1 billion by 2030 in the US alone.

No unanimously accepted approach has been established to date for the diagnosis of PJI. Abnormal concentrations of serum biomarkers and reduction in viscosity of synovial fluids have both been reported as criteria to evaluate the infection progression. However, the time required for these factors to reach levels sufficient for diagnosis can lead to further accumulation of pathogen and worsening infection. Further, current methods do not allow for the monitoring of local environmental changes for non-infectious inflammatory conditions or destructive processes that damage normal tissue and/or neoplasm. Therefore, a need still exists for a wireless implantable biosensor that can enable real-time, in situ detection of target bacteria during early stages of colonization and infection (e.g. 48-72 hours after surgery).

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

In one aspect of the present invention, a device for providing passive, wireless, in vivo detection of post-surgical infection in a surgical implant or prosthesis is provided. The device includes at least one magnetoelastic-based sensor associated with the implant or prosthesis. At least one magnetoelastic-based sensor is a differential sensor. Also, the differential sensor comprises a reference element and a sensing element.

In one embodiment, the reference element and the sensing element each have a length from about 0.1 mm to about 20 mm. In another embodiment, the reference element and the sensing element each have a length from about 9.5 mm to about 10 mm. In one embodiment, the reference element and the sensing element each have a width from about 0.01 mm to about 5 mm. In another embodiment, the reference element and the sensing element each have a width of about 1.5 mm. The two elements each have a thickness from about 0.01 mm to about 0.1 mm.

In one embodiment, the reference element and the sensing element have a length difference (ΔT) from about 0.1 mm to about 1 mm and a separation gap (g) from about 0.1 mm to about 5 mm. In one embodiment, the reference element and sensing element have a length difference (ΔL) of about 0.6 mm and a separation gap (g) of about 1.5 mm.

In another embodiment, the reference element and sensing element have shapes selected from the group consisting of triangular, hexagonal, circular, and rectangular. In one embodiment, the reference element and sensing element are both a triangular shape. In another embodiment, the surgical implant is an orthopedic implant.

In one embodiment, the at least one magnetoelastic-based sensor has a sensor surface, and further, wherein one or more bio-recognizers are immobilized on at least a portion of the sensor surface, wherein the bio-recognizers are selected from the group consisting of antibodies, aptamers, nucleic acids, and proteins, and further, wherein the bio-recognizers are capable of binding to one or more analytes, said analytes selected from the group consisting of pathogens, bacteria, virus, biomarkers, proteins, and nucleic acids. In another embodiment, the bio-recognizers are immobilized on all of the sensor surface.

In one embodiment, the bio-recognizers are antibodies immobilized on the sensor surface, said antibodies having antigen binding sites that are capable of binding with one or more post-surgical infectious bacteria. In another embodiment, the bacteria are selected from the group consisting of Escherichia coli, Staphylococcus aureus, Enterococcus spp, Pseudomonas aeruginosa, Klebsiella spp., Proteus spp., Citrobacter spp. and Coagulase-negative staphylococci.

In one embodiment, the bacteria are selected from the group consisting of Escherichia coli, Staphylococcus aureus, and Enterococcus spp. In another embodiment, the bacteria is Escherichia coli. In one embodiment, one or more linker molecules are immobilized on at least a portion of the sensor surface. In another embodiment, protein G is immobilized on at least a portion of the sensor surface. In one embodiment, one or more coupling microstructures are immobilized on at least a portion of the sensor surface. In another embodiment, the coupling microstructures are selected from the group consisting of gold nanoparticles, magnetic beads, nanotubes, and graphene. In one embodiment, one or more biomolecules are immobilized on at least a portion of the sensor surface.

In another embodiment, the reference element has a reference element surface and the sensing element has a sensing element surface, and further, wherein either the reference element surface, the sensing element surface, or both, comprise a coating that maintains detection performance of the sensor. In one embodiment, the reference element has a reference element surface and the sensing element has a sensing element surface, and further, wherein either the reference element surface, the sensing element surface, or both, comprise a coating that enhances compatibility of the sensor with a target environment of application. In another embodiment, the coating comprises an inert metal selected from the group consisting of gold, titanium, and chromium.

In one embodiment, the coating comprises a polymer selected from the group consisting of polyamides, parylene and combinations thereof. In another embodiment, the device also includes a package comprising the at least one magnetoelastic-based sensor, wherein the package is integrated with microfluidic features.

In another aspect of the present invention, a method of detecting a post-surgical infection is provided. The method involves implanting a magnetoelastic-based sensor associated with a surgical implant or prosthesis in a patient having surgery. At least one magnetoelastic-based sensor is a differential sensor and the differential sensor comprises a reference element and a sensing element. One or more bio-recognizers are immobilized on at least a portion of the sensor surface. The bio-recognizers are selected from the group consisting of antibodies, aptamers, nucleic acids, and proteins. Also, the bio-recognizers can bind to one or more analytes, and the analytes are selected from the group consisting of pathogens, bacteria, virus, biomarkers, proteins, and nucleic acids. The method further involves interrogating the sensor to determine the prevalence of analytes bound to the bio-recognizers, resulting in sensor output data. Finally, the sensor output data is used to determine the level of infection-related analytes.

In one embodiment, the sensor is interrogated by a coil in a location adjacent to the implanted sensor and external to a patient's body. In another embodiment, the coil is located in a coil patch. In one embodiment, the coil patch is connected to a unit that is wearable by the patient. The coil can be placed vertically or horizontally. The number of coil can be one or two.

In another aspect of the present invention a magnetoelastic-based sensor is provided. At least one magnetoelastic-based sensor is a differential sensor and the differential sensor comprises a reference element and a sensing element. Also, one or more bio-recognizers are immobilized on at least a portion of the sensor surface. The bio-recognizers are selected from the group consisting of antibodies, aptamers, nucleic acids, and proteins. In addition, the bio-recognizers are capable of binding to one or more analytes. The analytes are selected from the group consisting of pathogens, bacteria, virus, biomarkers, proteins, and nucleic acids.

### BRIEF DESCRIPTION OF THE DRAWINGS

The accompanying drawings, which are incorporated in and constitute a part of this specification, illustrate embodiments of the invention and, together with the general description of the invention given above, and the detailed description given below, serve to explain the principles of the invention.

FIG. 1A is an illustration showing an embodiment of the present invention for an ME biosensor used for in situ early detection of PJI.

FIG. 1B is an illustration showing an example application scenario of the ME sensor for PJI detection.

FIG. 1C is an illustration showing a prosthetic knee joint with an integrated biocompatible packaged sensor.

FIG. 1D is an illustration showing a biosensor functionalized with antibodies targeting specific types of bacteria.

FIG. 1E is an illustration showing a package configuration according to the present invention with a perforated lid.

FIG. 2A is an illustration showing a horizontal arrangement of a two coil configuration.

FIG. 2B is an illustration showing a vertical arrangement of a two coil configuration.

FIG. 2C is an illustration showing a single coil configuration.

FIG. 3 is an illustration showing a COMSOL Multiphysics simulation of a differential ME sensor. The selected gap and length difference between the two elements were g=1.5 mm and ΔL=0.6 mm, respectively. Smaller displacement observed for sensing element due to mass loading as expected.

FIG. 4A is a photo of a differential ME sensor (Metglas 2826 MB) according to the present invention.

FIG. 4B is a photo of a package and lid with anchors (3D printed from VisiJet M3 resin).

FIG. 4C is a photo of a fully assembled device according to the present invention.

FIG. 4D is a photo of a packaged device attached to a prosthetic knee joint, demonstrating one intended integration site.

FIG. 5 is a graph of measurement results showing the characterization of differential ME biosensor for mass detection (ink coating) in different media and the effectiveness of eliminating medium effect on sensor response.

FIG. 6A is an AFM image of bare gold surface.

FIG. 6B is an SEM image of a bare gold surface.

FIG. 6C is an AFM image of a surface after direct antibody immobilization without protein G.

FIG. 6D is an AFM image of a surface after antibody immobilization with protein G.

FIG. 6E is an SEM image of a surface after direct antibody immobilization without protein G.

FIG. 6F is an SEM image of a surface after antibody immobilization with protein G.

FIG. 6G is a fluorescence image of a surface after direct antibody immobilization without protein G

FIG. 6H is a fluorescence image of a surface after antibody immobilization with protein G.

FIG. 7 is a graph showing in vitro test results of differential sensors for E. coli detection.

FIG. 8A is a graph showing in vitro test results of differential sensors (g=1.5 mm, ΔL=0.6 mm) in E. coli suspensions with different viscosities (1-5.9 cP). The displayed data is raw data of measured Δf before differential correction.

FIG. 8B is a graph showing in vitro test results of differential sensors (g=1.5 mm, ΔL=0.6 mm) in E. coli suspensions with different viscosities (1-5.9 cP). The displayed data shows Δf after differential correction with an algorithm.

FIG. 9 is an illustration of different embodiments of device shapes for each element in a differential sensor configuration according to the present invention. A. Rectangle; B. Triangle; C. Rhombus; D. Hexagon; E. Circular.

FIG. 10 is an illustration of different embodiments of device arrangement methods for differential configuration according to the present invention. A. In serial; B. In parallel; C. In array.

FIG. 11A is an illustration of a differential configuration for application 1 of an ME biosensor according to the present invention.

FIG. 11B is an illustration of a differential configuration for application 2 of an ME biosensor according to the present invention.

FIG. 11C is an illustration of a differential configuration for application 3 of an ME biosensor according to the present invention.

FIG. 12A is a photo of microfluidic channels assembled on a package according to the present invention.

FIG. 12B is a functional block diagram of a microfluidic device as part of the sensor package.

FIG. 13 is a graph showing mass sensitivity of different sensor geometry and size as well as sensor with partial mass loading.

FIG. 14 is a graph showing in vitro test results using sensors with and without protein G treatment during sensor functionalization.

FIG. 15A is a graph showing the measurement results for Δf_r for dimensional optimization (gap and ΔL) of differential ME sensors.

FIG. 15B is a graph showing the measurement results for Δf_s for dimensional optimization (gap and ΔL) of differential ME sensors.

FIG. 15C is a graph showing the measurement results for δΔf_rs for dimensional optimization (gap and ΔL) of differential ME sensors.

FIG. 15D is an illustration of geometric parameters (gap and ΔL) of differential sensor configuration using triangular shape.

### DEFINITIONS

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

As used herein, the term “about,” when referring to a value or to an amount of mass, weight, time, volume, pH, size, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, “bio-recognizers” means a biological composition that can be immobilized on a sensor surface and can identify pathogens like bacteria and virus, biomarkers, proteins, nucleic acids, pH, temperature, or chemicals.

As used herein, “differential sensor” means a sensor with one or more coils that detects changes over time in the magnetic flux when detecting electrical voltages induced in the coil or coils. Since the change with time t can be described with the differential d/dt, the sensor is known as a “differential sensor”.

As used herein, “post-surgical infectious bacteria” means bacteria that are commonly known to infect a post-surgical site in humans.

While the following terms are believed to be well understood by one of ordinary skill in the art, definitions are set forth to facilitate explanation of the disclosed subject matter. Unless defined otherwise, all technical and scientific terms used herein have the same meaning as commonly understood by one of ordinary skill in the art to which the disclosed subject matter belongs.

### DETAILED DESCRIPTION OF THE INVENTION

One skilled in the art will recognize that the various embodiments may be practiced without one or more of the specific details described herein, or with other replacement and/or additional methods, materials, or components. In other instances, well-known structures, materials, or operations are not shown or described in detail herein to avoid obscuring aspects of various embodiments of the invention. Similarly, for purposes of explanation, specific numbers, materials, and configurations are set forth herein in order to provide a thorough understanding of the invention. Furthermore, it is understood that the various embodiments shown in the figures are illustrative representations and are not necessarily drawn to scale.

Reference throughout this specification to “one embodiment” or “an embodiment” means that a particular feature, structure, material, or characteristic described in connection with the embodiment is included in at least one embodiment of the invention, but does not denote that they are present in every embodiment. Thus, the appearances of the phrases “in an embodiment” or “in another embodiment” in various places throughout this specification are not necessarily referring to the same embodiment of the invention. Further, “a component” may be representative of one or more components and, thus, may be used herein to mean “at least one.”

The present invention involves implantable, passive wireless biosensors based on the magneto-elastic (ME) transduction mechanism with immobilized bio-recognizers (antibodies, aptamers, etc.) that specifically target certain pathogens, such as bacteria, and biomarkers. The device is intended for passive, wireless detection of post-surgical infections and other complications inside the body that would benefit from early recognition and continuous monitoring. Another aspect of the present invention is a method of integrating the biosensor with a surgical implant for passive wireless monitoring, thus allowing for continuous monitoring at any time point following implantation.

One application enabled by the device of the present invention is in-situ early detection of infection following total joint arthroplasty (periprosthetic joint infection, “PJI”). In these situations, the diagnosis of infection is often delayed to the point that resultant loosening and/or failure has already occurred. Late diagnosis of PJI necessitates more extensive and numerous invasive surgical treatments resulting in increased patient morbidity and financial cost. At present, there is no unanimously accepted standard approach or mechanism in place for early diagnosis of PJI.

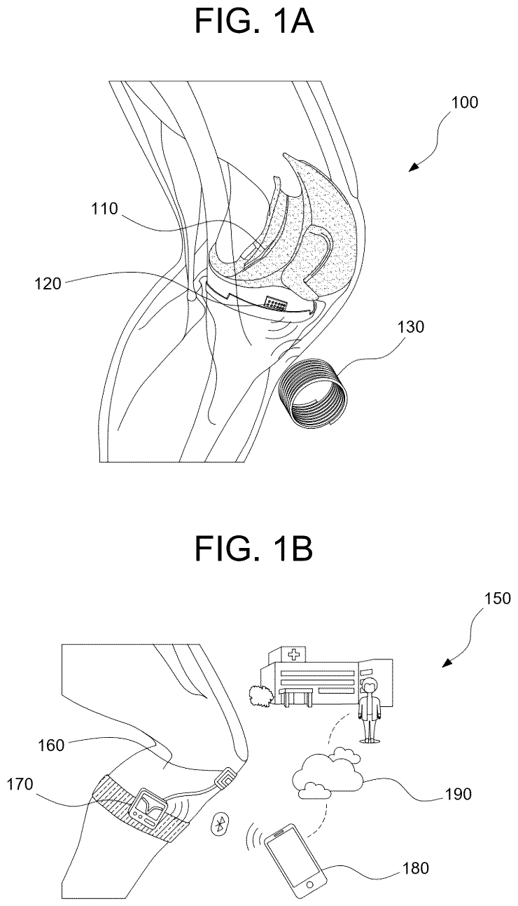

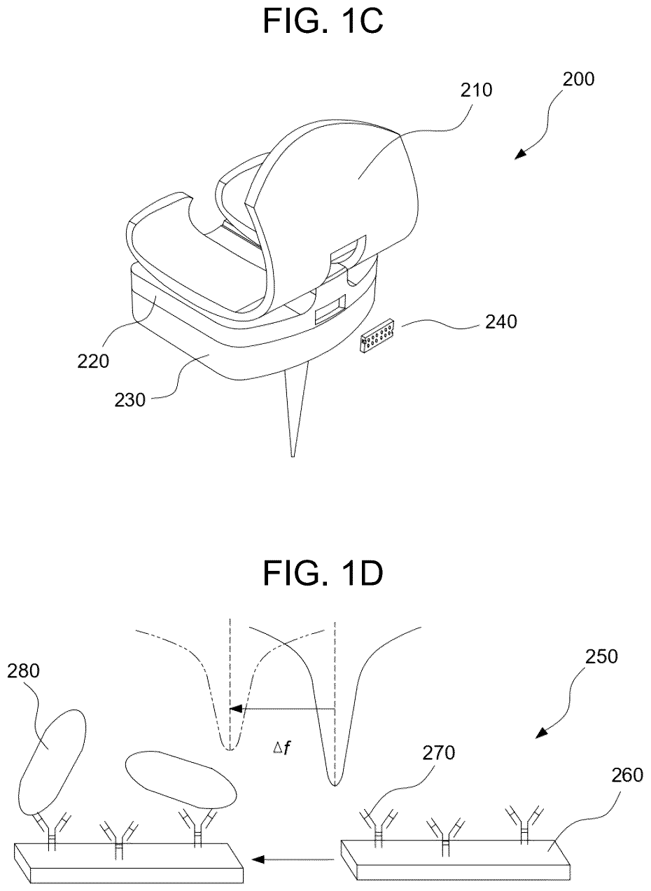

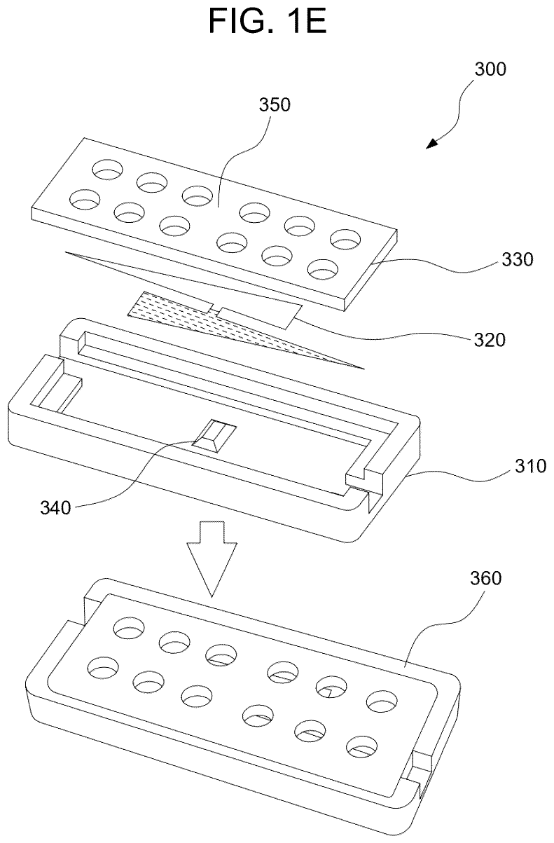

One embodiment of the present invention featuring PJI detection is illustrated in FIGS. 1A-1E. In this embodiment, the biosensor of the present invention is integrated with a knee arthroplasty implant to facilitate in situ early detection of PJI. The miniature ME sensor, contained within its own casing, is integrated onto the surface of, or alternatively, into a shallow recess formed on the surface of the knee replacement prosthesis. At any time following surgical implantation, an external coil patch can be placed on the knee in an adjacent location to the implanted sensor and connected to a small wearable unit to wirelessly excite and interrogate the sensor.

The wearable unit may have built-in alarms and user interface for patient interaction. It may also connect with a smartphone through a Bluetooth Low Energy (BLE) link, or other low power wireless communication methods, for remote monitoring and control. The sensor data can be uploaded by the smartphone through a cellular link to the cloud to enable remote access and telediagnosis with hospitals and doctors.

Apart from PJI, this ME sensor of the present invention has applications in monitoring for post-traumatic infection after treatment of open fractures if the device is implanted along with orthopedic fracture fixation devices such as intramedullary rods or fracture fixation plates/screws. In another embodiment, the present device is used to monitor local environmental changes for non-infectious inflammatory conditions, destructive processes that damage normal tissue, and/or neoplasm if idiosyncratic biomarkers specific to the pathology are targeted with the coating on the ME sensor.

The invention utilizes a magnetoelastic (ME) transduction mechanism combined with antibody-functionalization to target specific types of bacteria. Magnetoelastic sensors have been used in a wide range of applications such as the measurements of temperature, pressure, stress/strain, pH, metal ion concentration, cell growth, and concentrations of pathogen and biomarkers. The inherent passive wireless capability of ME sensors makes them highly desirable for implantable applications without the need for an antenna or local power source, such as their use with orthopedic implants to monitor potential structural failures and with biliary stents to monitor sludge accumulation and restenosis.

A novel differential sensor configuration is used in the present invention to distinguish the effects of target bacteria from variations caused by the surrounding fluid medium. In one embodiment, a triangular geometry is used for the sensor design, replacing traditional rectangular shapes used in ME immunosensors to enhance the mass sensitivity and facilitate early infection detection.

Functionalization of sensor surfaces with selected antibodies allows the sensor to target specific types of bacteria of interest. Bacteria of interest for post-surgical infection include Escherichia coli, Staphylococcus aureus, and Enterococcus spp. Other frequently identified microorganisms in infected post-operative wounds include Pseudomonas aeruginosa, Klebsiella spp., Proteus spp., Citrobacter spp. and coagulase-negative staphylococci.

### Device Design

FIGS. 1A-1E illustrate the concept of the present invention for an ME biosensor used for in situ early detection of PJI. Regarding FIG. 1A, a detection system 100 is shown. It involves a packaged sensor 120 integrated in a prosthetic knee joint 110. An external detection coil 130 interrogates the packaged sensor 120. Regarding FIG. 1B, an external support system 150 for a device according to the present invention is shown. An external coil patch 160 can be attached on the skin near the packaged sensor and connected to a wearable unit 170 to wirelessly interrogate the sensor. The wearable unit can be further connected wirelessly with a smartphone 180, allowing remote access and telediagnosis 190 through a cellular link. In another embodiment, the sensor is wirelessly interrogated using external coils that are interfaced with an external unit for sensor excitation and signal readout (see FIG. 1A).

Regarding FIG. 1C, a prosthetic knee joint 200 is shown. A packaged sensor 240 is integrated in the prosthetic knee joint 200. The prosthetic knee joint 200 further comprises a femoral component 210, a tibial tray 230 and a polymer bearing 220.

FIG. 1D shows a wireless biosensor system 250, in which an ME biosensor 260 is functionalized with antibodies 270 targeting specific types of bacteria 280. FIG. 1E shows a packaged sensor assembly 300. An ME biosensor 320 is mounted in a biocompatible package 310 for integration into a recess on a prosthetic knee joint (see FIGS. 1A and 1C). The package 310 has anchors 340 and 350 (located under the package lid 330) to suspend the biosensor 320 inside, preventing physical interference from tissue surrounding the implant area, while the perforations on the package lid 330 allow exchange of fluid (see FIG. 1E). The assembled full packaged sensor device is shown as 360.

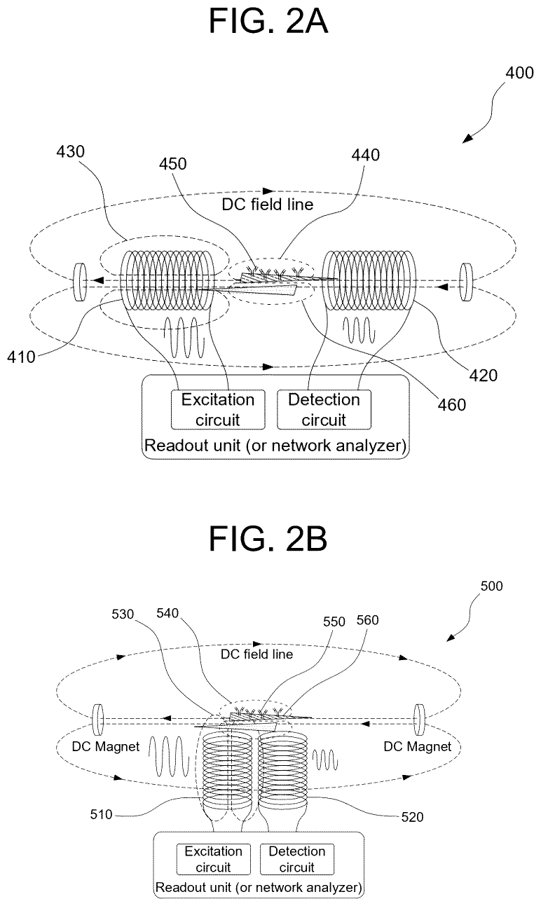

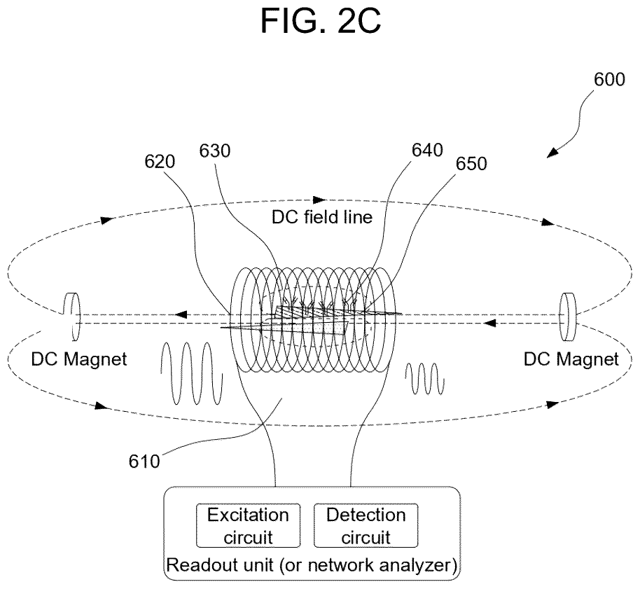

The working principle of the ME biosensor is illustrated in FIGS. 2A-2C, which show alternative embodiments of coil arrangements. When the ME sensor is excited by a time-varying magnetic field generated from a transmit coil, it produces a longitudinal vibration. This generates a magnetic flux with a resonance frequency, which can vary with changes in the boundary conditions of the sensor such as the mass and fluid medium in contact with the sensor. This flux can be detected wirelessly with a receive coil to measure the resonance frequency.

FIG. 2A shows a horizontal arrangement of a two-coil configuration sensor 400 with a transmit coil 410 and a receive coil 420. An ME biosensor 460 is located between the coils. The surface of the ME biosensor 460 comprises immobilized molecules 450. The transmit coil 410 produces an AC field line 430. The ME biosensor 460 produces a sensor field line 440. FIG. 2B shows an alternative coil design, a vertical arrangement of a two coil configuration sensor 500 with a transmit coil 510 and a receive coil 520. An ME biosensor 560 is located above the coils. The surface of the ME biosensor 560 comprises immobilized molecules 550. The transmit coil 510 produces an AC field line 530. The ME biosensor 560 produces a sensor field line 540.

Alternatively, as shown in FIG. 2C, a single coil can be used for both excitation and readout using signal reflection. The single coil configuration 600 involves a transmit/receive coil 610. An ME biosensor 650 is located inside the coil 610. The surface of the ME biosensor 650 comprises immobilized molecules 640. The transmit/receive coil 610 produces an AC field line 620. The ME biosensor 650 produces a sensor field line 630.

When a small mass, Δm, is applied to the ME sensor of an initial mass M, the resonance frequency shift, Δf, can be derived from equations given in as

$$Δ f = - \frac{Δ m}{4 LM} \sqrt{\frac{E}{ρ ( 1 - {v}^{2} )}} \qquad Equation 1$$

where L is the length of the sensor; E, ρ, and ν are Young's modulus, density, and Poisson's ratio for the ME material, respectively. When the properties of the fluid medium change, the corresponding Δf is given as

$$Δ f = - \frac{\sqrt{π {f}_{0}}}{2 π ρ d} \sqrt{η {ρ}_{m}} \qquad Equation 2$$

where f₀ is the resonance frequency in air, ρ and d are the density and thickness of the ME sensor, and ρₘ and η are the density and viscosity of the medium, respectively.

ME sensors have shown high performance as wireless immunosensors for in vitro applications in liquid media. Changes in the medium properties and conditions such as temperature, density, viscosity, and pH, can cause significant changes in the resonance frequency of the ME immunosensors; therefore, these parameters are usually controlled carefully to maintain the sensor performance for immunoassay under in vitro conditions. However, for the targeted in vivo application, the properties of the surrounding body fluid, particularly the viscosity and density, can change at any time. To eliminate the effect of the surrounding medium, a novel differential sensor consisting of two ME sensors is utilized: one with, and the other without functionalized antibodies. The element without functionalization is used as a reference. When target bacteria are present in the medium and become bound to the antibodies, mass loading is applied only to the sensing element and any common mode changes such as those of the medium properties are eliminated by subtracting the outputs of the sensing and reference elements.

Mass sensitivity is defined as the frequency shift caused by a unit amount of mass loading. Higher mass sensitivity is desirable to provide a larger frequency shift for a given amount of mass loading, which is particularly important for the detection of bacteria during early stages of infection when the bacteria concentration is relatively low. It has been shown previously that the geometry of a ME sensor can affect its magnetic domain distribution; geometries with sharp corners can result in small magnetic domains, leading to stronger vibration and higher sensitivity. In one embodiment of the present invention, triangular geometry is used for the sensor design instead of the traditional rectangular geometry that has been commonly used for ME immunosensors.

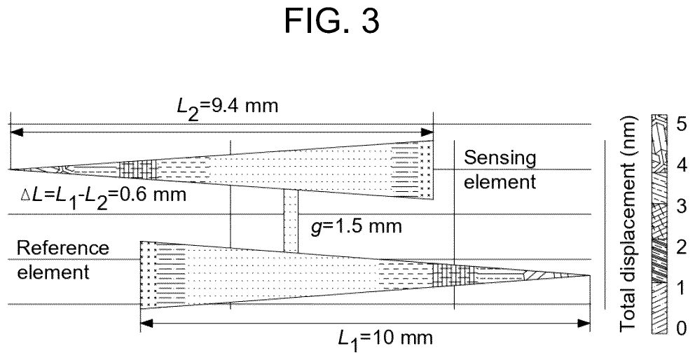

A triangular geometry, instead of the traditional rectangular geometry commonly used for ME immunosensors, is used in an embodiment of the present invention to provide a higher mass sensitivity. Effort has gone into the design and optimization of the differential sensor configuration. Particularly, to minimize the magnetic coupling and thus the interference between the two elements, a separation gap g and a length difference ΔL between the two elements are critical parameters of the differential design. FIG. 3 shows an example geometric design of the differential ME sensor and the simulation results obtained using COMSOL® Multiphysics. An example design with ΔL=0.6 mm and g=1.5 mm was found to provide adequately low coupling of magnetic flux between the two elements. The anchor of each element is placed near the null point of vibration of the geometry.

### Device Fabrication

The ME differential sensors were fabricated from ribbons of Metglas® 2826 MB (Fe₄₅Ni₄₅Mo₇B₃) alloy from Metglas Inc. using an in-house high-precision micro-electro-discharge machine (Smaltec® EM203 μEDM). The sensing element was coated on one side with a Cr/Au layer (40/60 nm thick) by e-beam evaporation while the reference element was protected from deposition with photoresist. The Au surface provides a critical, biocompatible layer for antibody immobilization while Cr serves as an adhesion layer.

Surface functionalization included forming a self-assembled monolayer (SAM) on the Au surface using cysteamine (CYSTE), immobilizing protein G on the SAM via the N-(3-Dimethylaminopropyl)-N′-ethylcarbodiimide (EDC) and N-hydroxysulfosuccinimide (Sulfo-NHS) protocol, incubating antibodies to bind with protein G on the sensors, and finally treating with bovine serum albumin (BSA) to block non-specific binding sites. Protein G is used as a linker molecule to achieve orthogonal antibody immobilization, allowing higher antigen capture rates and greater antibody binding density. For this work, lyophilized cells of strain K12 E. coli, Sulfo-NHS, protein G and BSA were purchased from Sigma Aldrich; CYSTE (98%), phosphate buffered saline (PBS, pH 7.4), rabbit anti-E. coli polyclonal antibody, goat anti-rabbit antibody conjugated with Alexa Fluo® 488, and EDC were acquired from Fisher Scientific.

The sensor functionalization procedure began with thorough cleaning of the ME sensors in an ultrasonic cleaner using acetone, isopropanol, and DI water, sequentially. The sensors were then immersed in a CYSTE solution (10 mM) for 16 h to deposit the SAM. Protein G (2 μg/mL) was activated in a solution containing EDC (0.01 mM) and Sulfo-NHS (0.02 mM) for 1 h at 37° C. After rinsing with PBS, the ME sensors were soaked in an activated protein G solution for 2 h at 37° C. Another PBS rinsing step was done to remove loosely-bonded protein G. This was followed by incubating the sensors in an activated antibody solution for 2 h at 37° C. to immobilize the anti-E. coli antibodies. Loosely-bonded antibodies were removed by another PBS rinsing. The sensors were then treated with a 1% w/w BSA solution for 30 min, rinsed with PBS and dried under a nitrogen stream to become ready for testing.

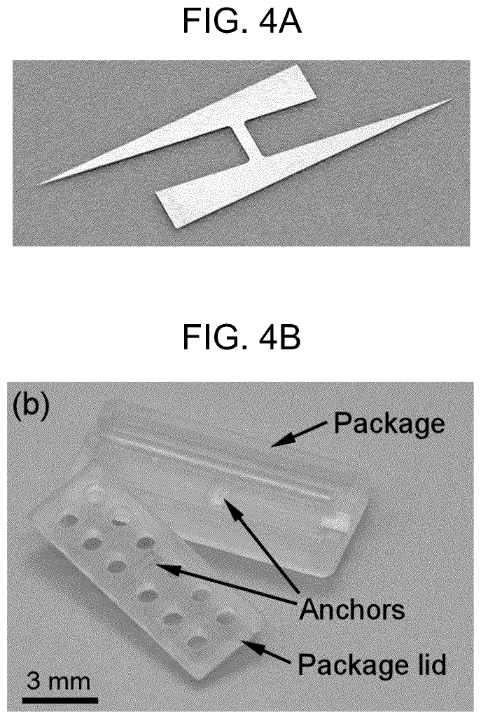

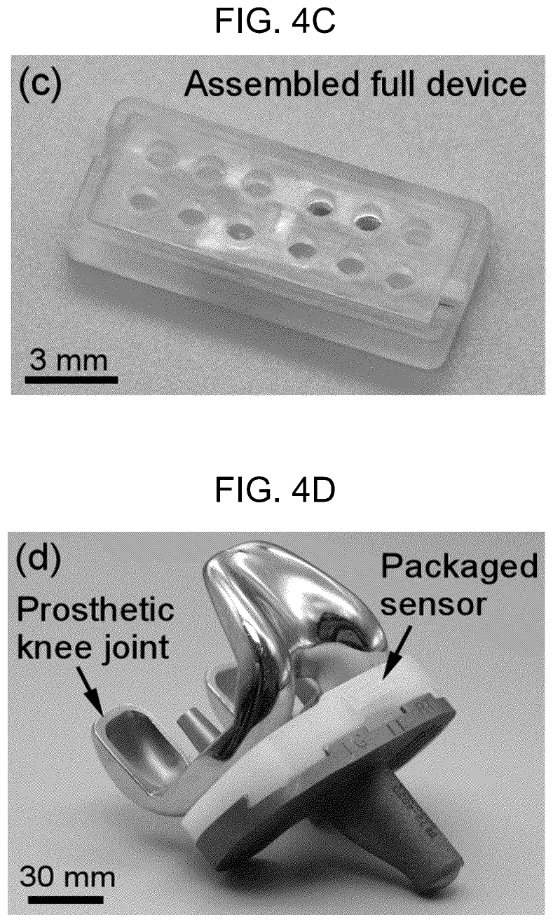

The sensor package with the perforated lid was 3D printed from a biocompatible resin (VisiJet® M3). Two anchors in the package are used to clamp the joint area of the differential sensor and suspend it in the package for free vibration (FIG. 4). The packaged sensor can then be integrated in a recess on the prosthetic knee joint.

### ME Biosensor Geometry and Arrangement in Differential Configuration

The ME sensor of the present invention can be used in either a single or differential configuration. While a single ME sensor is capable of converting analyte concentration into a frequency shift for wireless detection and interrogation, a differential configuration is essential for proper detection of target analytes in the in vivo environment. In such environment, properties of the surrounding fluid, such as temperature, pH, density, and viscosity, are continuously subject to change, thus causing interference on the sensor output. With the differential configuration, one or more reference elements that respond only to the effects of the surrounding fluid are used to generate a baseline signal. This baseline can be subtracted from the signal of the sensing elements that respond to the analytes in addition to the fluid effects, thus providing an output that correlates only to the target analytes. Each of the two or more elements in the differential sensor configuration can use one of the geometries shown in FIG. 9, and can also vary in dimension (length, width, thickness) as well as relative arrangements (in parallel, in serial, in array, etc.) (FIG. 10) to optimize the cancellation effect.

### Variations in Bio-Recognizer Configuration

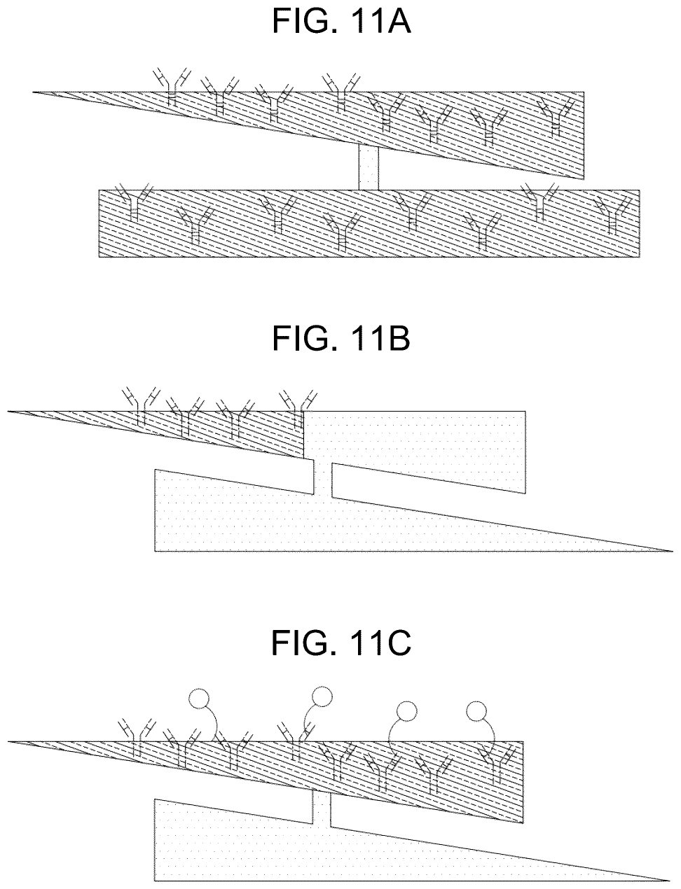

The characteristics of the bio-recognizing layer immobilized on the surface of the sensor are an important element as they determine the amount of mass loading to the ME biosensor, and directly affect the sensor functionality and performance. In order to impart functionality, the bio-recognizers can include, but are not limited to, antibodies, aptamers, nucleic acids, and proteins that can target specific or multiple analytes. One or more bio-recognizers can be immobilized concurrently on the surface of the ME biosensor to permit simultaneous detection of multiple analytes and measurands: pathogens like bacteria and virus, biomarkers, proteins, nucleic acids, pH, temperature, chemicals, etc. (FIG. 11A).

Several methods for sensitivity enhancement and improvement can be implemented. Bio-recognizers can be immobilized on either the entire sensor surface or only on selected regions of the surface (FIG. 11B). Partial loading of only selected regions of the sensor surface with a bio-recognizing layer has been shown to improve the mass sensitivity of ME sensors. Some biomarkers such as proteins, nucleic acids, and small molecules are found in only trace levels, particularly in the early stages of disease progression. Coupling microstructures (such as gold nanoparticles, magnetic beads, nanotubes, graphene, etc.) and biomolecules (such as enzymes, precipitations, etc.) to the analyte have been shown to significantly enhance the mass loading effect of the bio-recognizing layer and can be used as an amplification method to improve the detection limit with lower analyte concentrations. (FIG. 11C). Combinations of these strategies could potentially produce synergistic effects that further enhance the performance of the sensors.

### Package Design and Integration in the Implant

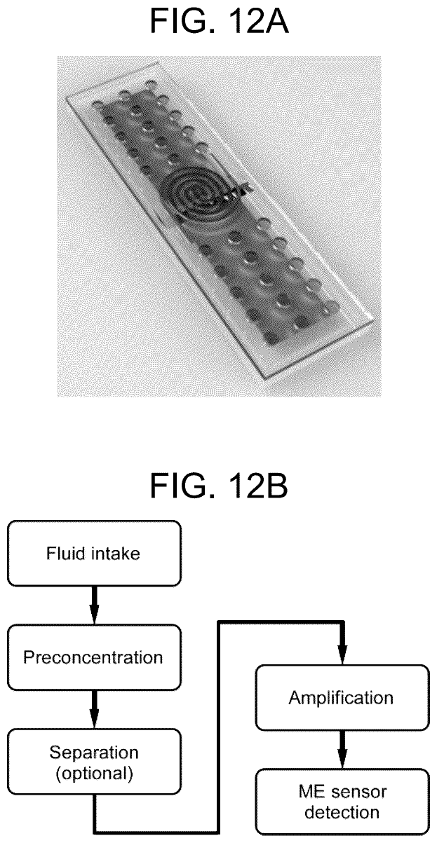

In many embodiments, the ME biosensors of the present invention are contained within a compact, biocompatible package that can be produced by 3D printing, injection molding, and other precision manufacturing techniques. The package prevents physical interference from surrounding tissue while allowing exchange of fluid through perforations or channels around the package structure (FIG. 1E). Further, microfluidic channels can be assembled on the packages to enable functions such as preconcentration, separation, amplification and analysis — similar to those available in “lab-on-a-chip” devices (FIG. 12A). An example functional block diagram of the microfluidic device is shown in FIG. 12B.

One or more packages can be integrated into one or more shallow recesses on the implant or fracture fixation device. The physical location of biosensor integration onto the implant can be on any surface that does not negatively impact the performance or functions of the implant, while maintaining direct fluid exchange with the local tissue environment.

### Readout Coil Configurations

In some embodiments, external coils are used to excite and then detect the resultant signals from ME sensors. Various coil configurations with different wire gauge, number of turns, diameter, number of coils, and coil orientation, can be used to enhance the sensor interrogation quality and performance. For example, different orientations of coil placement relative to the sensor (such as horizontal, FIG. 2A, or vertical, FIG. 2B) can be implemented. Alternatively, a single coil can be used for both excitation and readout using signal reflection (FIG. 2C).

### EXAMPLES

As shown in the examples below, in vitro tests for proof of concept were carried out using single sensors in both rectangular and triangular shapes in E. coli suspension and PBS control solution, and successfully demonstrated a 2.63× improvement in mass sensitivity for the triangular sensors. Differential operation of the sensor was successfully tested for mass detection in various media (air, water, standard 5 cSt fluid, and paraffin oil), demonstrating effective elimination of medium effect by subtracting reference sensor outputs.

### Example 1

Two 40-turn coils of a ¾ inch diameter made from 28 AWG magnet wire were used as the transmit and receive coils, respectively. A network analyzer (Keysight® E5061B) was connected to the two coils for sensor interrogation. The power of the excitation output from the network analyzer was 5 dBm and the received signal was processed by the network analyzer to generate the frequency response and thus the resonance frequency of the ME sensors.

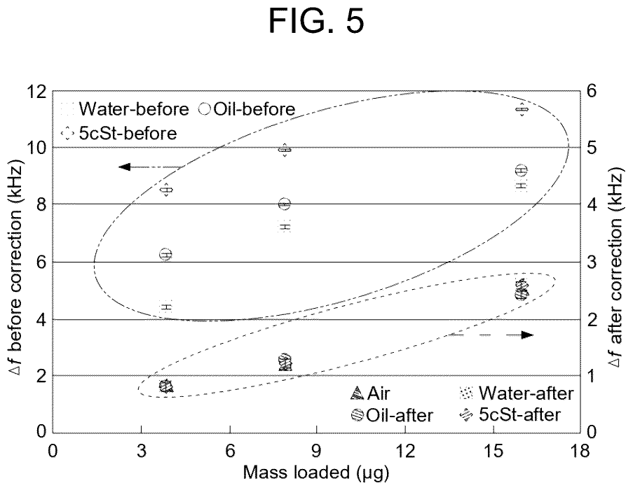

Experiments were performed to verify the differential operation of the ME sensor and its capability to effectively eliminate the effect of varying medium properties. For these tests, mass loading was applied by coating multiple layers of ink on the sensing element of the differential sensors. The sensors were first tested in air to generate the baseline response and then tested in three different media (water, standard 5 cSt fluid, and paraffin oil) to emulate the impact of varying density and viscosity of the medium. The reference element outputs were scaled to correct for the frequency difference caused by the length mismatch in the sensor design, and then subtracted from the sensing element outputs to cancel the medium effect. As demonstrated by the measurement results in FIG. 5, the same mass loading generated different frequency shifts in various media before correction (data points inside the red dashed circle). This can lead to serious discrepancy when interpreting the frequency response caused by mass loading. By subtracting the scaled outputs from the reference element in each medium, the medium effect was effectively eliminated (data points inside the lower dashed circle), demonstrating the validity of the differential sensor mechanism.

### Example 2

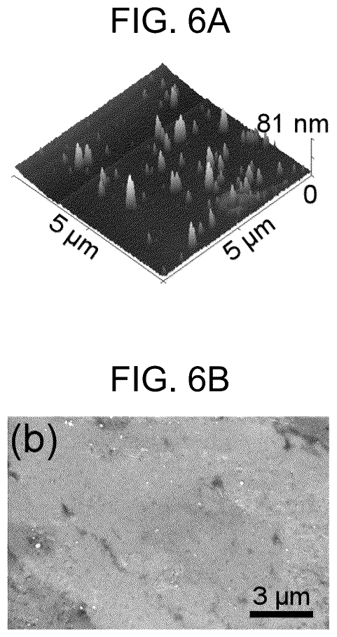

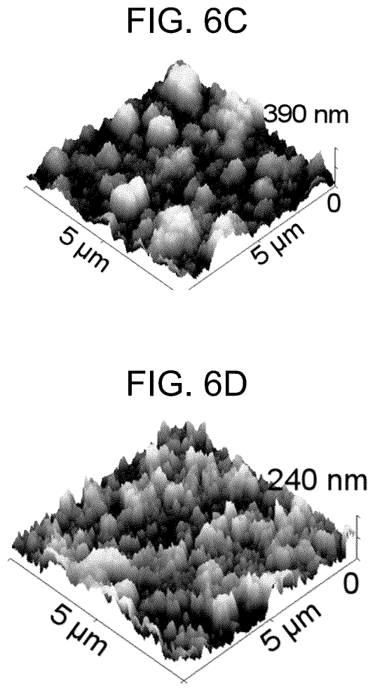

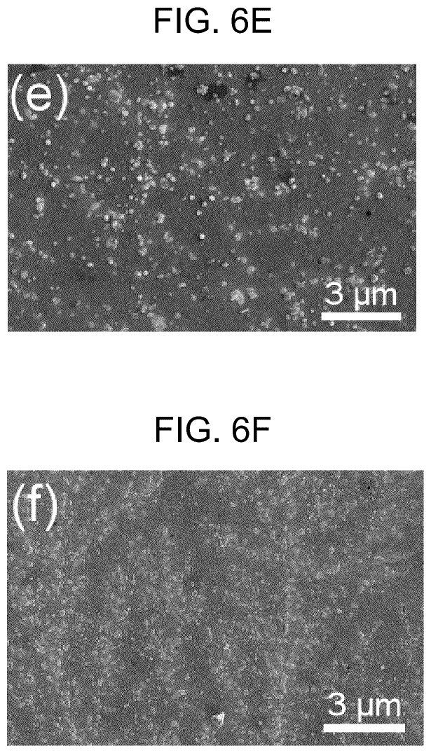

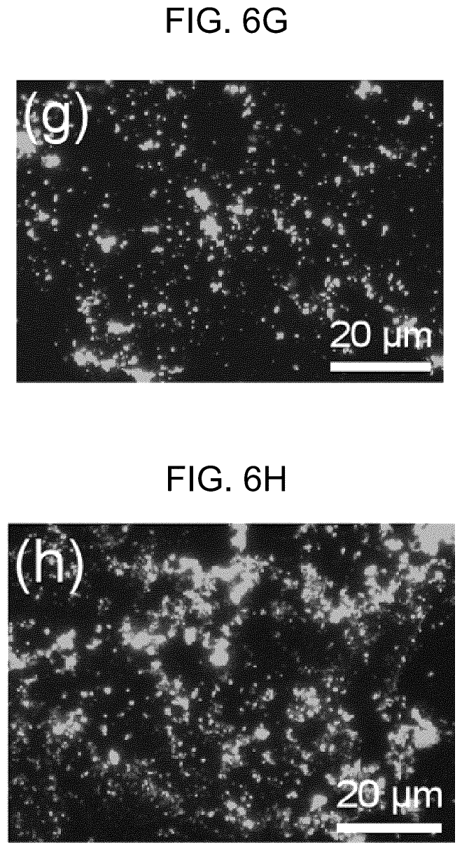

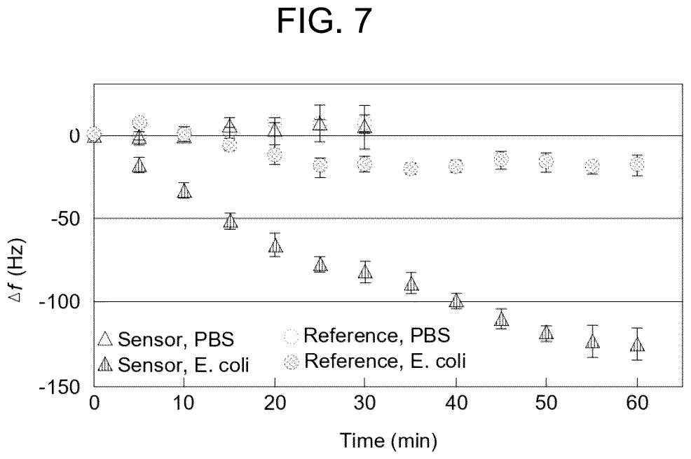

Imaging techniques including scanning electron microscopy (SEM), atomic force microscopy (AFM) and fluorescence microscopy were used to evaluate the performance of surface functionalization with or without the use of protein G. Fluorescence images obtained using secondary antibodies (goat anti-rabbit antibody conjugated with Alexa Fluor 488, 4 μg/mL) with the Olympus IX81 microscope verified an improved antibody coverage when protein G was used (FIGS. 6G and 6H). SEM images showed higher antibody binding density with the use of protein G (FIGS. 6E and 6F). AFM images showed the different surface roughness with and without protein G (FIGS. 6C and 6D).

All sensor experiments described below were performed using a 50-turn coil of ¾-inch diameter made from 28 AWG magnet wire. The coil was connected to a network analyzer (Keysight® E5061B) for extraction of the resonance frequencies of the ME sensors.

The feasibility of the differential sensor for in vitro bacterial detection was validated experimentally. Functionalized sensors were first tested in 1 mL PBS solution for 30 min as the control. Then 10 μL E. coli suspension was added to the PBS solution and the test continued for another 60 min. The concentration of E. coli in the solution was 5×10⁶ cfu/mL as determined by plate count. The test was repeated four times using different sensors with results averaged (N=4) and shown in FIG. 7. Regarding FIG. 7, the data was averaged from readings of 4 sensors (N=4). Error bar shows standard error. The sensing element had increasing Δf magnitude with E. coli binding, while Δf of reference remained close to 0.

During the control period in PBS, the responses of both sensing and reference elements were stable at about zero. After the bacteria were added, the sensing element showed increasing Δf magnitude due to E. coli binding, while the Δf of the reference element had a slight increase and then remained stable. The slight increase may be caused by minor change in the viscosity of the solution when the bacteria suspension was added.

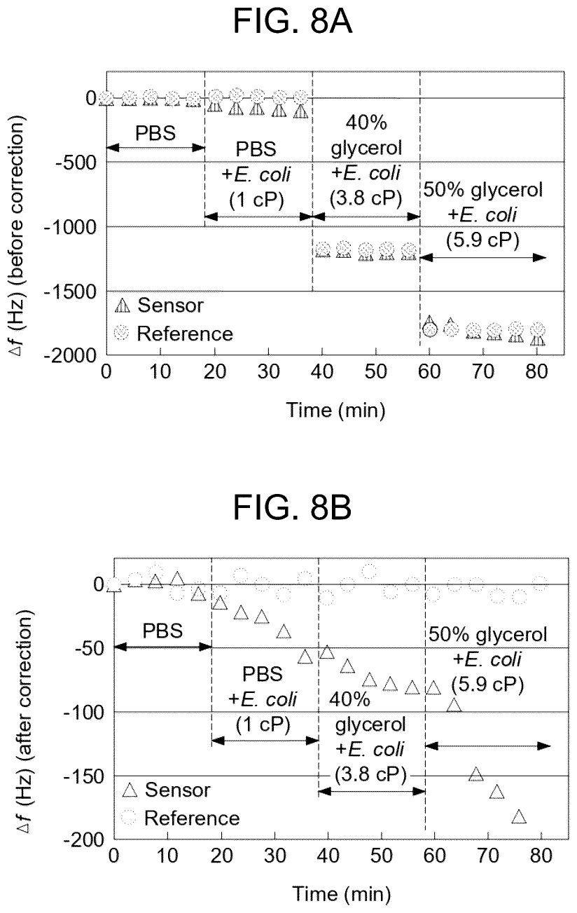

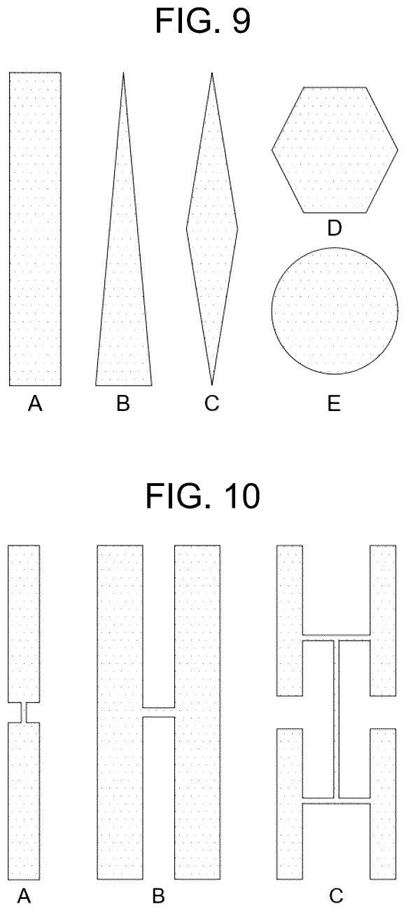

The capability of the differential sensor to eliminate the effect of varying medium properties were also verified by in vitro tests. The sensors were first tested in PBS solution for 20 min as the control, and then sequentially in three E. coli suspensions with different medium for 20 min each to emulate the impact of varying medium properties. The three E. coli suspensions were made with PBS as well as 40% and 50% glycerol/water solutions, resulting in viscosity of 1 cp, 3.8 cp and 5.9 cp, respectively as determined by NDJ-55 rotational viscometer. As shown in FIGS. 8A and 8B, a large Δf corresponding to the change in medium properties can lead to significant error when interpreting the sensor response attributable to mass loading from bacteria binding. After differential correction with an algorithm, the corrected Δf of the reference element stayed close to zero while the corrected Δf of the sensing element gradually increased in magnitude as expected. This validated the differential sensor mechanism by demonstrating effective elimination of medium effect.

Regarding FIGS. 8A and 8B, the figures show in vitro test results of differential sensors (g=1.5 mm, ΔL=0.6 mm) in E. coli suspensions with different viscosities (1-5.9 cP). The sensor was tested in PBS control solution for 20 min, and then 3 types of E. coli suspensions (5×10⁶ cfu/mL) for 20 min each. (a) Raw data of measured Δf before differential correction, showing large Δf for both sensing and reference elements due to changes in the medium properties. (b) Δf after differential correction with an algorithm, showing corrected Δf of the reference element stayed close to 0 while that of the sensing element gradually increased as expected, demonstrating effective elimination of medium effect using the differential operation.

### Example 3

The effects of changing device size and geometry as well as using the partial mass loading approach on mass sensitivity were evaluated through experiments. Mass loading was applied by depositing a 50 nm thick Cr layer through the e-beam evaporation, and partial mass loading on selected region of the sensor surface was done by coving the undesired area with tape during the evaporation. The resonance frequency shift of the sensors was generated by measuring the resonance frequencies of the sensor before and after the deposition using a network analyzer and then calculating the difference.

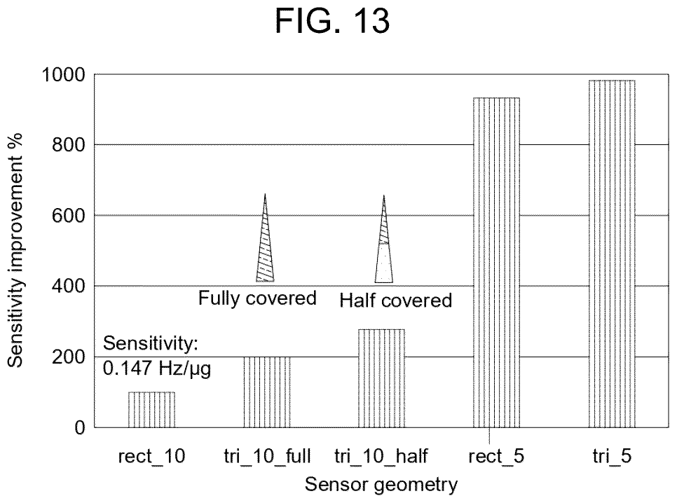

As shown in FIG. 13, the triangular device has better sensitivity compared to the rectangular device with the same equivalent size (base and height). The half-loading triangular device (mass covering half-length from the tip of the triangular sensor) shows larger mass sensitivity than the triangular device with full mass loading. With decreased sensor size, increased mass sensitivity was observed.

Regarding FIG. 13, the graph shows mass sensitivity of different sensor geometry and size as well as sensor with partial mass loading. The sensor varieties include (from left to right): 10 mm rectangular sensor with full mass loading (rect_10), 10 mm triangular sensor with full mass loading (tri_10_full), 10 mm triangular sensor with half mass loading (tri_10_half), 5 mm rectangular sensor with full mass loading (rect_5), and 5 mm triangular sensor with full mass loading (tri_5).

### Example 4

Larger frequency shift is desired to help improve sensor performance. This, in turn, requires more amount of the target analyte attached to the sensor surface to provide larger amount of mass loading. A straightforward way to increase the amount of attached analyte is to increase the bio-recognizer density. Protein G (prG) has high affinity to specifically bind with the Fc region of an antibody. This allows the antibody to be bound with a proper orientation that exposes the antigen binding sites of the antibody to the target solution, significantly improving the detection efficiency. Therefore, the use of prG is added to the surface functionalization procedure.

In vitro tests were conducted to demonstrate the effect of using prG. Two of the 10 mm rectangular ME sensors were deposited with 50 nm Cr followed by 80 nm Au. The same functionalization procedure was applied to both sensors except that one sensor was treated with prG solution for 1 hour at room temperature before incubating with antibody.

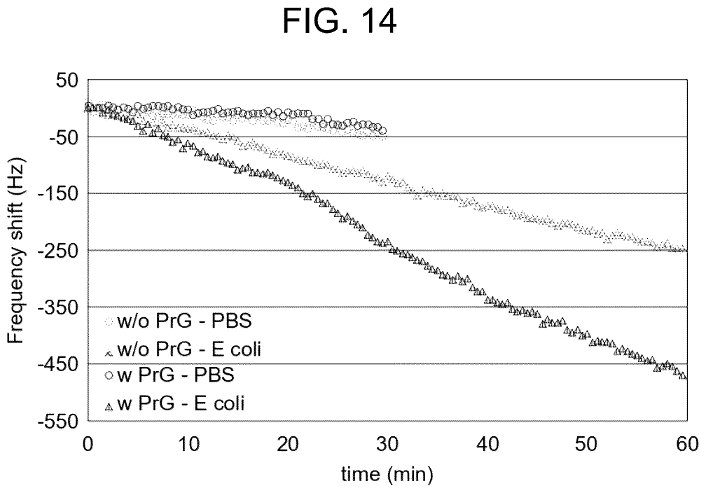

As shown in FIG. 14, both sensors with and without the prG treatment show stable response in PBS as control. When adding E. coli suspension, larger frequency shift was observed for the sensor with the prG treatment, verifying the performance improvement by using prG in sensor functionalization.

Regarding FIG. 14, the graph shows in vitro test results using sensors with and without protein G treatment during sensor functionalization. In both cases stable response was observed in PBS for control. When adding E. coli suspension, larger frequency shift was observed for the sensor with protein G treatment,

### Example 5

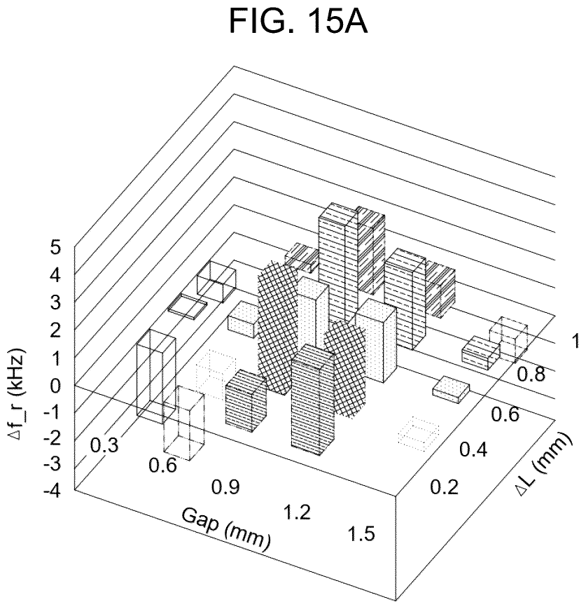

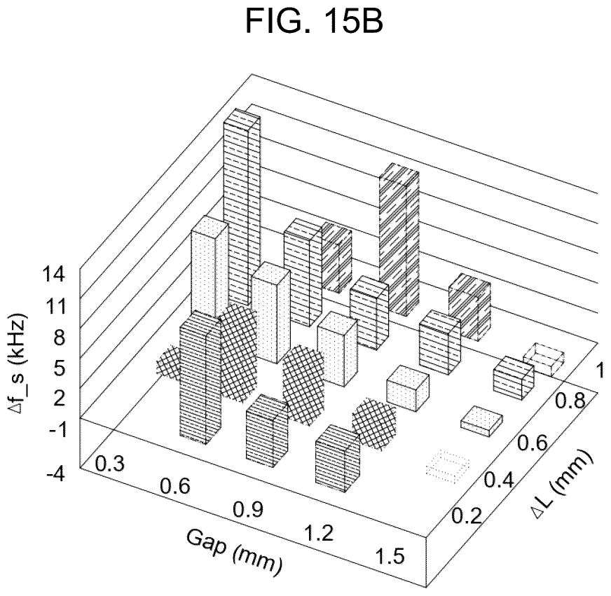

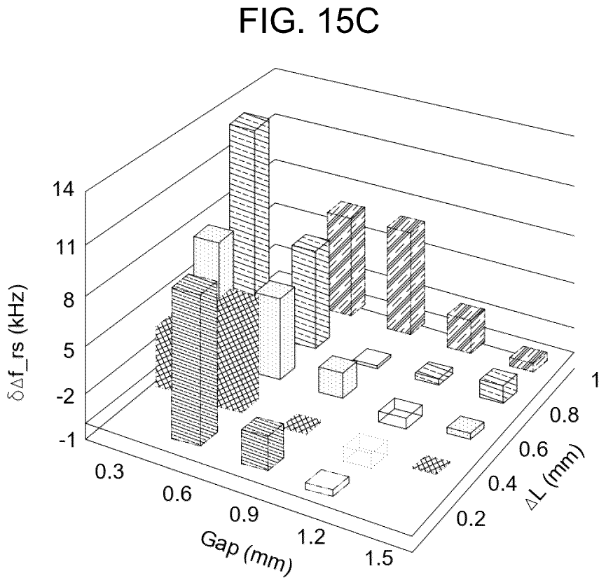

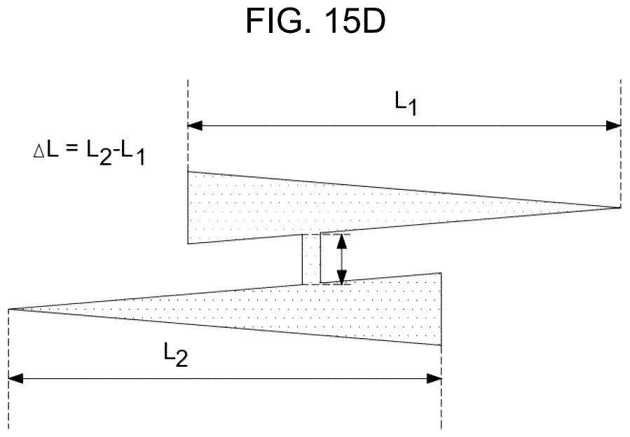

The differential sensor configuration is essential for the target in vivo applications. There are two parameters that significantly affect the performance of the differential ME sensor: the gap between the two elements and the length difference (ΔL) of them. A group of differential ME sensors with varying values of the gap and ΔL was fabricated by micro electro-discharge machining (micro-EDM) and tested to optimize the values of these two parameters. The frequencies of the two resonance peaks of each differential ME sensor were read by a network analyzer. The longer element which has a lower resonance frequency was defined as the reference element, while the shorter element with a higher frequency was defined as the sensing element. Because of the magnetic coupling between the two elements, the two resonance peaks of the differential sensor can shift compared to two single sensors with the same lengths as the reference and sensing elements, respectively. The resonance frequency shifts of the reference and sensing elements due to the magnetic coupling are noted as Δf_r and Δf_s, respectively. The distance of the two resonance peaks of the differential sensor is noted as Δf_rs. The difference of Δf_rs between a differential sensor and two single sensors with the same lengths as the reference and sensing elements, respectively, is noted as δΔf_rs. FIG. 15A shows Δf_r, FIG. 15B shows Δf_s, and FIG. 15 C shows δΔf_rs of the differential sensors with the gap ranging from 0.3 mm to 1.5 mm and ΔL ranging from 0.2 mm to 1 mm. The sensing elements have higher frequency shift, suggesting that they were affected by the magnetic coupling more than the reference elements. Sensors with larger gaps show smaller δΔf_rs, indicating less coupling between the two elements. FIG. 15D shows geometric parameters (gap and ΔL) of differential sensor configuration using triangular shape as an example.

The results demonstrate a method that can be used to optimize and select the values of the gap and ΔL to minimize the effect of magnetic coupling between the two elements of the differential ME sensor. This can be used to guide the design and selection of the differential configuration. Generally, larger values of gap and ΔL can benefit the differential sensor performance due to less magnetic coupling between the two elements; the two parameters can also affect each other and need to be considered and optimized at the same time.

As shown above, a novel implantable differential biosensor with passive wireless interrogation capability has been designed to facilitate in situ early detection of PJI. Functionalized with selected antibodies, the sensor can target specific types of bacteria known to be present during the early stages of PJI. Improved performance of functionalization was achieved by using protein G. Wireless differential operation of the sensor for bacterial detection was successfully tested in vitro, in PBS and in E. coli suspensions with viscosity ranging between 1-5.9 cP, demonstrating effective elimination of the medium effect.

All documents cited are incorporated herein by reference; the citation of any document is not to be construed as an admission that it is prior art with respect to the present invention.

It is to be further understood that where descriptions of various embodiments use the term “comprising,” and/or “including” those skilled in the art would understand that in some specific instances, an embodiment can be alternatively described using language “consisting essentially of” or “consisting of.”

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A device for providing in vivo detection of post-surgical infection in a surgical implant or prosthesis, said device comprising at least one magnetoelastic-based sensor associated with said implant or prosthesis, wherein at least one magnetoelastic-based sensor is a differential sensor, and further, wherein the differential sensor comprises a reference element and a sensing element.

2. The device of claim 1 wherein the reference element and the sensing element each have a length from about 0.1 mm to about 20 mm.

3. The device of claim 1 wherein the reference element and the sensing element each have a width from about 0.01 mm to about 5 mm, and further, wherein the reference element and the sensing element each have a thickness from about 0.01 mm to about 0.1 mm.

4. The device of claim 1 wherein the reference element and the sensing element have a length difference (ΔL) from about 0.1 mm to about 1 mm and a separation gap (g) from about 0.1 mm to about 5 mm.

5. The device of claim 1 wherein the reference element and sensing element have a length difference (ΔL) of about 0.6 mm and a separation gap (g) of about 1.5 mm.

6. The device of claim 1 wherein the reference element and sensing element have shapes selected from the group consisting of triangular, hexagonal, circular, and rectangular.

7. The device of claim 1 wherein the reference element and sensing element are both a triangular shape.

8. The device of claim 1 wherein the surgical implant is an orthopedic implant.

9. The device of claim 1 wherein the at least one magnetoelastic-based sensor has a sensor surface, and further, wherein one or more bio-recognizers are immobilized on at least a portion of the sensor surface, wherein the bio-recognizers are selected from the group consisting of antibodies, aptamers, nucleic acids, and proteins, and further, wherein the bio-recognizers are capable of binding to one or more analytes, said analytes selected from the group consisting of pathogens, bacteria, virus, biomarkers, proteins, and nucleic acids.

10. The device of claim 9 wherein the bio-recognizers are immobilized on all of the sensor surface.

11. The device of claim 9 wherein the bio-recognizers are antibodies immobilized on the sensor surface, said antibodies having antigen binding sites that are capable of binding with one or more post-surgical infectious bacteria.

12. The device of claim 11 wherein the bacteria are selected from the group consisting of Escherichia coli, Staphylococcus aureus, Enterococcus spp, Pseudomonas aeruginosa, Klebsiella spp., Proteus spp., Citrobacter spp. and Coagulase-negative staphylococci.

13. The device of claim 11 wherein the bacteria are selected from the group consisting of Escherichia coli, Staphylococcus aureus, and Enterococcus spp.

14. The device of claim 11 wherein the bacteria is Escherichia coli.

15. The device of claim 11 wherein one or more linker molecules are immobilized on at least a portion of the sensor surface.

16. The device of claim 11 wherein protein G is immobilized on at least a portion of the sensor surface.

17. The device of claim 11 wherein one or more coupling microstructures are immobilized on at least a portion of the sensor surface.

18. The device of claim 17 wherein the coupling microstructures are selected from the group consisting of gold nanoparticles, magnetic beads, nanotubes, and graphene.

19. The device of claim 11 wherein one or more biomolecules are immobilized on at least a portion of the sensor surface.

20. The device of claim 1 wherein the reference element has a reference element surface and the sensing element has a sensing element surface, and further, wherein either the reference element surface, the sensing element surface, or both, comprise a coating that maintains detection performance of the sensor.

21. The device of claim 1 wherein the reference element has a reference element surface and the sensing element has a sensing element surface, and further, wherein either the reference element surface, the sensing element surface, or both, comprise a coating that enhances compatibility of the sensor with a target environment of application.

22. The device of claim 20 wherein the coating comprises an inert metal selected from the group consisting of gold, titanium, aluminum, and chromium.

23. The device of claim 21 wherein the coating comprises an inert metal selected from the group consisting of gold, titanium, chromium and other metals and alloys.

24. The device of claim 20 wherein the coating comprises a polymer selected from the group consisting of polyamides, Parylene, hydrogel, polyethylene glycol, polyethyleneimine and combinations thereof.

25. The device of claim 21 wherein the coating comprises a polymer selected from the group consisting of polyamides, Parylene and combinations thereof.

26. The device of claim 1 further comprising a package comprising the at least one magnetoelastic-based sensor, wherein the package is integrated with microfluidic features.

27. A method of detecting a post-surgical infection comprising:
a. implanting a magnetoelastic-based sensor associated with a surgical implant or prosthesis in a patient having surgery, wherein at least one magnetoelastic-based sensor is a differential sensor, wherein the differential sensor comprises a reference element and a sensing element; wherein said sensor has a sensor surface, and further, wherein one or more bio-recognizers are immobilized on at least a portion of the sensor surface, wherein the bio-recognizers are selected from the group consisting of antibodies, aptamers, nucleic acids, and proteins, and further, wherein the bio-recognizers are capable of binding to one or more analytes, said analytes selected from the group consisting of pathogens, bacteria, virus, biomarkers, proteins, and nucleic acids;
b. interrogating the sensor to determine the prevalence of analytes bound to the bio-recognizers, resulting in sensor output data and
c. using the sensor output data to determine the level of infection-related analytes.

28. The method of claim 27 wherein the sensor is interrogated by a coil in a location adjacent to the implanted sensor and external to a patient's body.

29. The method of claim 28 wherein the coil is located in a coil patch.

30. The method of claim 29 wherein the coil patch is connected to a unit that is wearable by the patient.

31. A magnetoelastic-based sensor, said sensor having a sensor surface, wherein at least one magnetoelastic-based sensor is a differential sensor, wherein the differential sensor comprises a reference element and a sensing element, wherein one or more bio-recognizers are immobilized on at least a portion of the sensor surface, wherein the bio-recognizers are selected from the group consisting of antibodies, aptamers, nucleic acids, and proteins, and further, wherein the bio-recognizers are capable of binding to one or more analytes, said analytes selected from the group consisting of pathogens, bacteria, virus, biomarkers, proteins, and nucleic acids.

32. The device of claim 31 wherein the reference element and sensing element are both a triangular shape.

33. The device of claim 31 wherein the bio-recognizers are antibodies immobilized onto the sensor surface, said antibodies having antigen binding sites that are capable of binding with one or more post-surgical infectious bacteria.
